(19) United States
(12) Patent Application Publication
(10) Pub. No.: US 2025/0011451 A1
(43) Pub. Date: Jan. 9, 2025

(54) FUSION PROTEINS FOR IMMUNOTHERAPY AGAINST CANCER AND INFECTIOUS DISEASES

(71) Applicant: NAVICURE BIOPHARMACEUTICALS LIMITED, Apia City (WS)

(72) Inventor: Chia-Mao WU, Hsinchu City (TW)

(21) Appl. No.: 18/826,183

(22) Filed: Sep. 6, 2024

Related U.S. Application Data
(63) Continuation of application No. 17/240,160, filed on Apr. 26, 2021.
(60) Provisional application No. 63/020,545, filed on May 6, 2020.

(51) Int. Cl. C07K 16/28; A61P 35/00

(52) U.S. Cl. CPC C07K 16/2878; A61P 35/00; C07K 2317/565; C07K 2317/622; C07K 2319/01; C07K 2319/55

(57) ABSTRACT

Fusion proteins for immunotherapy against cancer and infectious diseases are disclosed. A fusion protein according to the invention comprises a CD40-binding domain; an antigen; and a translocation domain located between the CD40-binding domain and the antigen, in which a furin and/or cathepsin L cleavage site is present in the fusion protein between the CD40-binding domain and the translocation domain. The antigen is an antigen of a pathogen or a tumor antigen. The furin and/or cathepsin L cleavage site permits removal of the CD40-binding domain away from the fusion protein via furin and/or cathepsin L cleavage. Also disclosed are pharmaceutical compositions, expression vectors and use of the fusion proteins of the invention for eliciting an antigen-specific cell-mediated immune response, treating a tumor and/or a disease caused by a pathogen in a subject in need thereof.

### REFERENCE TO RELATED APPLICATION

The present application is a continuation of and claims the priority to U.S. Ser. No. 17/240,160, filed Apr. 26, 2021, which status is pending and claims priority to U.S. Provisional Application Ser. No. 63/020,545, filed May 6, 2020, all of which are herein incorporated by reference in their entireties.

### REFERENCE TO AN ELECTRONIC SEQUENCE LISTING

The contents of the electronic sequence listing (10040-001PCT_sequence listing_ST26_ 20241001 updated.xml; size 79 KB; creation date Oct. 1, 2024) are herein incorporated by reference in their entireties.

### FIELD OF THE INVENTION

The present invention relates generally to fusion proteins, and more specifically to fusion proteins for eliciting T cell-mediated immune responses against tumors and infectious diseases.

### BACKGROUND OF THE INVENTION

The adaptive immune system includes both humoral immunity components and cell-mediated immunity components and destroys invading pathogens. The cells that carry out the adaptive immune response are white blood cells known as lymphocytes. B cells and T cells, two different types of lymphocytes, carry out the main activities: antibody responses, and cell-mediated immune response. The adaptive immunity is activated by exposure to pathogens and leads to an enhanced immune response to future encounters with that pathogen. Vaccines induce antigen-specific memory in adaptive immune cells that enables protection against the target pathogen. There is still a need for novel therapeutic vaccines to treat diseases including cancer and infectious diseases caused by pathogens.

### SUMMARY OF THE INVENTION

In one aspect, the invention relates to a fusion protein comprising: (a) a CD40-binding domain; (b) an antigen; and (c) a translocation domain located between the CD40-binding domain and the antigen; wherein a furin and/or cathepsin L cleavage site is present in the fusion protein between the CD40-binding domain and the translocation domain.

In another aspect, the invention relates to a DNA fragment encoding a fusion protein according to the invention. The invention also relates to an expressing vector comprising a DNA fragment encoding a fusion protein of the invention.

Further in another aspect, the invention relates to a pharmaceutical composition comprising a fusion protein of the invention and a pharmaceutical acceptable carrier and/or an adjuvant.

Yet in another aspect, the invention relates to a method for eliciting an antigen-specific cell-mediated immune response, comprising administering a therapeutically effective amount of the fusion protein of the invention to a subject in need thereof, and thereby eliciting an antigen-specific cell-mediated immune response in the subject in need thereof.

The invention also relates to a method for treating a tumor in a subject in need thereof, comprising administering to the subject in need thereof a therapeutically effective amount of the fusion protein of the invention, wherein the antigen of the fusion protein is a tumor antigen, and thereby treating the subject in need thereof.

The invention also relates to a method for treating a disease caused by a pathogen in a subject in need thereof, comprising administering to the subject in need thereof a therapeutically effective amount of the fusion protein of the invention, wherein the antigen of the fusion protein is an antigen of the pathogen, and thereby treating the disease caused by the pathogen.

### BRIEF DESCRIPTION OF THE DRAWINGS

FIG. 1 is a vector map.

FIG. 2 is a vector map. MCS, multiple cloning sites.

FIG. 3 is a vector map.

FIG. 4 is a vector map.

FIGS. 5A-E are schematic drawings illustrating various embodiments of the invention.

FIG. 6 is a graph showing relative cytokine inductions in each animal group.

FIG. 7 is a graph showing IFN-γ⁺ immunospots in the splenocytes from each animal group.

FIG. 8 is a graph showing serum HPV₁₆ E7-specific antibody level in each animal group.

FIG. 9 is a graph showing serum HPV₁₈ E7-specific antibody level in each animal group.

FIG. 10 shows an immunization schedule and animal groups treated and untreated with the indicated fusion proteins, respectively.

FIG. 11 is a graph showing tumor size in each animal group treated or untreated with the fusion protein indicated.

FIG. 12 is a graph showing survival rate in each animal group treated or untreated with the fusion protein indicated.

FIG. 13 is a graph showing tumor free rate in each animal group treated or untreated with the fusion protein indicated.

FIG. 14 is a graph showing tumor size in each animal group treated or untreated with the fusion protein 18sCD40L-T^{PE}-E7 at various doses indicated.

FIG. 15 is a graph showing tumor size in each animal group treated or untreated with the fusion protein E7-T^{Stx}-18sCD40L at various doses indicated.

FIG. 16 shows an immunization scheme (upper panel), animal groups and respective dosing schedules (lower panel) of the fusion protein HBx-preS1-T^{Stx}-18sCD40L.

FIG. 17 is a graph showing IFN-γ⁺ immunospots in the splenocytes from each animal group in FIG. 16.

FIG. 18 is a graph showing serum HBx-specific antibody level in cach animal group in FIG. 16.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

### Definition

An antigen-presenting cell (APC) or accessory cell is a cell that displays antigen complexed with major histocompatibility complexes (MHCs) on their surfaces; this process is known as antigen presentation. T cells may recognize these complexes using their T cell receptors (TCRs). APCs process antigens and present them to T cells.

Antigen-presenting cells fall into two categories: professional and non-professional. Those that express MHC class II molecules along with co-stimulatory molecules and pattern recognition receptors are called professional antigen-presenting cells. The main types of professional antigen-presenting cells are dendritic cells (DCs), macrophages and B cells. The non-professional APCs express MHC class I molecules, which include all nucleated cell types in the body.

Professional APCs specialize in presenting antigens to T cells. They are very efficient at internalizing antigens, either by phagocytosis, or by receptor-mediated endocytosis, processing the antigen into peptide fragments and then displaying those peptides (bound to a class II MHC molecule) on their membrane. The T cell recognizes and interacts with the antigen-class II MHC molecule complex on the membrane of the antigen-presenting cell. An additional co-stimulatory signal is then produced by the antigen-presenting cell, leading to activation of the T cell. All professional APCs also express MHC class I molecules as well.

Professional APCs and non-professional APCs use an MHC class I molecule to display endogenous peptides on the cell membrane. These peptides originate within the cell itself, in contrast to the exogenous antigen displayed by professional APCs using MHC class II molecules. Cytotoxic T cells are able to interact with antigens presented by the MHC class I molecule.

CD40 is a costimulatory protein expressed on antigen-presenting cells (c.g., dendritic cells, macrophages and B cells). The binding of CD40L to CD40 activates antigen-presenting cells and induces a variety of downstream effects. CD40 is a drug target for cancer immunotherapy.

The term “a CD40-binding domain” refers to a protein that can recognize and binds to CD40. A CD40-binding domain may be selected from one of the following: “CD40 ligand (CD40L) or a functional fragment thereof”, “an anti-CD40 antibody or a functional fragment thereof.”

The terms “CD40L”, “CD40 ligand” and “CD154” are interchangeable. CD40L binds to CD40(protcin) on antigen-presenting cells (APC), which leads to many effects depending on the target cell type. CD40L plays a central role in co-stimulation and regulation of the immune response via T cell priming and activation of CD40-expressing immune cells. U.S. Pat. No. 5,962,406 discloses the nucleotide and amino acid sequence of CD40L.

The terms “anti-CD40 antibody” and “CD40-specific antibody” are interchangeable.

When the term “consist substantially of” or “consisting substantially of” is used in describing an amino acid sequence of a polypeptide, it means that the polypeptide may or may not have a starting amino acid “M” (translated from a start codon AUG) at N-terminal as a part of the polypeptide, depending on protein translation requirements. For example, when the antigen HPV 18E7 protein (SEQ ID NO: 39) fused to another polypeptide (c.g., another antigen), the starting amino acid “M” could be omitted or kept.

As used herein, “a translocation domain” is a polypeptide having biological activity in translocating an antigen within a fusion protein across an endosomal membrane into the cytosol of the CD40-expressing cell. The translocation domain guides or facilitates the antigen toward class I major histocompatibility complex (MHC-1) pathway (i.c., a cytotoxic T cell pathway) for antigen presentation.

The term “a Pseudomonas Exotoxin A (PE) translocation peptide (T^{PE})” refers to a PE domain II peptide or a functional fragment thereof that has the biological activity in translocation.

The term “a Shiga toxin (Stx) translocation peptide (T^{Stx})” refers to a Stx translocating domain or a functional fragment thereof that has the biological activity in translocation.

The terms “furin and/or cathepsin L” or “furin/cathepsin L” are interchangeable. A furin and/or cathepsin L cleavage site refers to a protease (furin and/or cathepsin L) sensitive site. It is a short peptide sequence that can be cleaved by furin or cathepsin L, or by both furin and cathepsin L. It may be a peptide linker comprising said cleavage site that is introduced into the fusion protein, or an intrinsic protease cleavage site present in the translocation domain of the fusion protein.

The terms “antigen” and “immunogen” are interchangeable. An antigen refers to an antigenic protein, which may be a tumor antigen (an antigen from a cancer or an antigen associated with a cancer), or an antigen of a pathogen (an antigen from a pathogen).

The terms “tumor” and “cancer” are interchangeable.

The terms “an antigen of a cancer cell” and “a tumor antigen” are interchangeable.

The term “a tumor antigen” refers to a tumor-specific antigen and/or a tumor-associated antigen. A tumor-associated antigen may be a protein or polypeptide expressed on the surface of a tumor cell.

Cluster of Differentiation 28 (CD28) is a T-cell-specific surface glycoprotein. A CD28 receptor is stimulated during the contact of T cells with antigen-presenting cells. Its function is involved in T-cell activation, the induction of cell proliferation and cytokine production and promotion of T-cell survival.

The term “an effective amount” refers to the amount of an active fusion protein that is required to confer a therapeutic effect on the treated subject. Effective doses will vary, as recognized by those skilled in the art, depending on rout of administration, excipient usage, and the possibility of co-usage with other therapeutic treatment.

The term “treating”, or “treatment” refers to administration of an effective amount of the fusion protein to a subject in need thereof, who has cancer or infection, or a symptom or predisposition toward such a disease, with the purpose of cure, alleviate, relieve, remedy, ameliorate, or prevent the disease, the symptoms of it, or the predisposition towards it. Such a subject can be identified by a health care professional based on results from any suitable diagnostic method.

By “0 to 12 repeats” or “2 to 6 repeats”, it means that all integer unit amounts within the range “0 to 12” or “2 to 6” are specifically disclosed as part of the invention. Thus, 0, 1, 2, 3, 4, . . . 10, 11and 12″ or “2, 3, 4, 5 and 6” units amounts are included as embodiments of this invention.

In one aspect, the invention relates to a fusion protein comprising: (i) a CD40-binding domain; (ii) an antigen; and (iii) a translocation domain, located between the CD40-binding domain and the antigen, wherein a furin and/or cathepsin L cleavage site is present in the fusion protein between the CD40-binding domain and the translocation domain.

The fusion proteins of the invention can elicit an antigen-specific T cell immune response via

MHC class I antigen presentation pathway. They share a common mechanism of action. Using the fusion protein 18sCD40L-T^{PE}-E7 as an example, the mechanism of action is illustrated below:

(1) the fusion protein binds to a CD40-expressing cell (e.g., dendritic cell or macrophage) and is internalized via a CD40-mediated endocytosis;

(2) the fusion protein is cleaved by furin protease and/or cathepsin L protease within the endosome so as to remove the 18sCD40L fragment away from the T^{PE}-E7 fragment;

(3) the T^{PE}-E7 fragment is translocated across the endosomal membrane of the endosome into the cytosol;

(4) the T^{PE}-E7 fragment is digested by cytosol proteasome to generate small E7 antigens with epitopes;

(5) the E7 antigens are delivered to MHC class I pathway for antigen presentation; and

(6) a CD8+ T cell specific immune response is induced or enhanced by T-cell recognizing these presented antigens.

The above mechanism of action is applicable to the fusion protein E7-T^{Stx}-18sCD40L, in which case the furin and/or cathepsin L protease cleavage removes the E7-T^{Stx} fragment away from the 18sCD40L fragment. Thus, the E7-T^{Stx} fragment is translocated across the endosomal membrane of the endosome into the cytosol, digested by cytosol proteasome to generate small E7 antigens with epitopes; the E7 antigens delivered to MHC class I pathway for antigen presentation; and a CD8+ T cell specific immune response is induced or enhanced by T-cell recognizing these presented antigens.

According to the invention, no furin and/or cathepsin L cleavage site is present in the fusion protein between the antigen and the translocation domain.

The presence of the furin and/or cathepsin L cleavage site and its location in the fusion protein permits removal of the CD40-binding domain from the fusion protein after furin and/or cathepsin L cleavage.

In one embodiment, the fusion protein of the invention further comprises a peptide linker, said linker comprising the furin and/or cathepsin L cleavage site present in the fusion protein between the CD40-binding domain and the translocation domain.

The translocation domain and the antigen are located within the fusion protein in such an orientation and/or relation that permits the translocating domain to translocate the antigen across the membrane of the endosome and enter the cytosol, and then facilitate the antigen toward MHC class I pathway for antigen presentation in the CD40-expressing cell.

In one embodiment, the translocation domain is derived from a Pseudomonas Exotoxin A (PE). In another embodiment, the translocation domain is derived from a Shiga toxin (Stx).

In one embodiment, the translocation domain comprises or is a Pseudomonas Exotoxin A (PE) translocation peptide (T^{PE}), with the proviso that the CD40-binding domain is located at the N-terminal of the fusion protein.

In another embodiment, the translocation domain comprises or is a Shiga toxin (Stx) translocation peptide (T^{Stx}), with the proviso that the antigen is located at the N-terminal of the fusion protein.

In another embodiment, a fusion protein of the invention sequentially comprises: (i) a CD40-binding domain located at the N-terminal of the fusion protein; (ii) a translocation domain comprising a PE translocation peptide (T^{PE}); and (iii) an antigen located at the C-terminal of the fusion protein; wherein a furin and/or cathepsin L cleavage site is present in the fusion protein between the CD40-binding domain and the translocation domain.

In another embodiment, the translocation domain is a functional moiety of T^{PE} and the furin and/or cathepsin L cleavage site is an intrinsic furin cleavage site from PE.

In another embodiment, a fusion protein of the invention sequentially comprises: (i) a CD40-binding domain located at the N-terminal of the fusion protein; (ii) a peptide linker comprising a furin and/or cathepsin L cleavage site; (iii) a translocation domain comprising a PE translocation peptide (T^{PE}); and (iv) an antigen of a pathogen or a tumor antigen.

In another embodiment, a fusion protein of the invention sequentially comprises: (i) an antigen located at the N-terminal of the fusion protein; (ii) a translocation domain comprising a Stx translocation peptide (T^{Stx}); and (iii) a CD40-binding domain; wherein a furin and/or cathepsin L cleavage site is present in the fusion protein between the CD40-binding domain and the translocation domain.

In one embodiment, the translocation domain is a functional moiety of T^{Stx}, and said furin and/or cathepsin L cleavage site is an intrinsic furin cleavage site from Stx.

Further in another embodiment, a fusion protein of the invention sequentially comprises: (i) an antigen located at the N-terminal of the fusion protein; (ii) a translocation domain comprising a Stx translocation peptide (T^{Stx}); (iii) a cleavable linker comprising a furin and/or cathepsin L cleavage site; and (iv) a CD40-binding domain.

In one embodiment, a furin and/or cathepsin L cleavage site comprises, or is, or consists of, the amino acid sequence of SEQ ID NO: 1 or 2.

In another embodiment, a PE translocation peptide (T^{PE}) is the domain II (amino acid residues 253-364; SEQ ID NO: 9) of Pseudomonas Exotoxin A protein (full-length PE, SEQ ID NO: 4).

In another embodiment, a PE translocation peptide (T^{PE}) comprises the minimal functional fragment GWEQLEQCGYPVQRLVALYLAARLSW (SEQ ID NO: 5).

In another embodiment, a PE translocation peptide (T^{PE}) consists of 26-112 amino acid residues in length, said the PE translocation peptide comprises a minimal functional fragment of GWEQLEQCGYPVQRLVALYLAARLSW (SEQ ID NO: 5).

In another embodiment, a PE translocation peptide (T^{PE}) comprises an amino acid sequence that is at least 90%, 95% or 99% identical to SEQ ID NO: 5, 6, 7, 8 or 9.

In another embodiment, a PE translocation peptide (T^{PE}) is selected from the group consisting of PE₂₈₀₋₃₀₅ (SEQ ID NO: 5), PE₂₈₀₋₃₁₃ (SEQ ID NO: NO: 6), PE₂₆₈₋₃₁₃ (SEQ ID NO: NO: 7), PE₂₅₃₋₃₁₃ (SEQ ID NO: 8), and PE₂₅₃₋₃₆₄ (SEQ ID NO: 9; full-length PE domain II).

In one embodiment, a Stx translocation peptide (T^{Stx}) is a functional fragment of Shiga toxin (Stx) subunit A (SEQ ID NO: 10) or Shiga-like toxin I (Slt-I) subunit A (SEQ ID NO: 11). According to the invention, a Stx translocation peptide has translocation function but no cytotoxic effect of subunit A. Sequence identify between Shiga toxin (Stx) subunit A and Slt-I subunit A is 99% and the two proteins has only one amino acid difference.

In another embodiment, a Stx translocation peptide (T^{Stx}) consists of 8-84 amino acid residues in length.

In another embodiment, a Stx translocation peptide (T^{Stx}) comprises a minimal functional fragment of LNCHHHAS (SEQ ID NO: 12).

In another embodiment, a Stx translocation peptide (T^{Stx}) consists of 8-84 amino acid residues in length, said T^{Stx} comprising a minimal fragment of LNCHHHAS (SEQ ID NO: 12).

In another embodiment, a Stx translocation peptide (T^{Stx}) comprises an amino acid sequence that is at least 90%, 95% or 99% identical to the amino acid sequence selected from the group consisting of SEQ ID NOs: 12, 13, 14, 15 and 16.

In another embodiment, a Stx translocation peptide comprises an amino acid sequence selected from the group consisting of SEQ ID NOs: 12, 13, 14, 15 and 16.

In another embodiment, a Stx translocation peptide (T^{Stx}) is selected from the group consisting of Stx₂₄₀₋₂₄₇ (SEQ ID NO: 12), Stx₂₄₀₋₂₅₁ (SEQ ID NO: 13), Stx₂₁₁₋₂₄₇ (SEQ ID NO: 14), Stx₂₁₁₋₂₅₁ (SEQ ID NO: 15) and Stx ₁₆₈₋₂₅₁ (SEQ ID NO: 16) of Stx subunit A.

A CD40-binding domain is a polypeptide having biological activity in binding to CD40 protein on a CD40-expressing cell. A CD40-binding domain permits a fusion protein of the invention to bind to a CD40 receptor on a CD40-expressing cell (e.g., dendritic cell or macrophage).

In one embodiment, a CD40-binding domain is selected from the group consisting of (i) a CD40 ligand (CD40L) or a functional fragment thereof; and (ii) an anti-CD40 antibody or a functional fragment thereof.

The CD40L, the anti-CD40 antibody, and the respective functional fragments thereof all have biological activity in binding to CD40 protein on a CD40-expressing cell. A functional fragment of CD40L is a truncated CD40L with biological activity, substantially lacking transmembrane and cytoplasmic regions of the full-length CD40L1-261 protein (SEQ ID NO: 17).

In another embodiment, a CD40L or a functional fragment thereof consists of 154-261 amino acid residues in length.

In another embodiment, a truncated CD40L with functional activity is selected from the group consisting of CD40L₄₇₋₂₆₁ (SEQ ID NO: 18) and CD40L₁₀₈₋₂₆₁ (named 18sCD40L; SEQ ID NO: 19).

In another embodiment, a CD40 ligand (CD40L) or a functional fragment thereof consists of 154-261 amino acid residues in length, said functional fragment thereof comprises CD40L₁₀₈₋₂₆₁ (SEQ ID NO: 19).

In another embodiment, a CD40L comprises or consists of an amino acid sequence that is at least 90%, 95% or 99% identical to SEQ ID NO: 17, 18 or 19, said CD40L having biological activity in binding to CD40 protein on a CD40-expressing cell.

In another embodiment, a CD40 ligand (CD40L) is selected from the group consisting of CD40L₄₇₋₂₆₁ (SEQ ID NO: 18), CD40L₁₀₈₋₂₆₁ (SEQ ID NO: 19; referred to as 18sCD40L) and CD40L₁₋₂₆₁ (SEQ ID NO: 17).

In another embodiment, a CD40-binding domain is a CD40-specific antibody (or anti-CD40antibody). A CD40-specific antibody is an antibody specific against CD 40 protein. A CD40-specific antibody can bind to CD40 protein on a CD40-expressing cell.

In one embodiment, the CD40-specific antibody comprises a heavy chain variable domain (V_{H}) and a light chain variable domain (V_{L}), wherein the V_{H} comprises the amino acid sequence of SEQ ID NO: 22; and the V_{L} comprises the amino acid sequence of SEQ ID NO: 23.

In another embodiment, the CD40-specific antibody according to the invention is selected from the group consisting of a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab₂, a minibody and a fully antibody.

In another embodiment, a CD40-binding domain according to the invention is a CD40-specific scFv (anti-CD40 scFv) comprising a heavy chain variable domain (V_{H}), a light chain variable domain (V_{L}) and a flexible linker (L) connecting the V_{H} and the V_{L}.

In one embodiment, a CD40-specific scFv comprises the amino acid sequence of SEQ ID NO: 20 or 21.

In another embodiment, the CD40-binding domain according to the invention is (i) a CD40-specific antibody or a binding fragment thereof, or (ii) a CD40-specific single chain variable fragment (scFv) or a binding fragment thereof; said CD40-specific antibody or said CD40-specific scFv comprising a V_{H} and a V_{L}, wherein: (a) the V_{H} comprises the amino acid sequence of SEQ ID NO: 22; and (b) the V_{L} comprises the amino acid sequence of SEQ ID NO: 23.

In another embodiment, the CD40-specific antibody or CD40-specific scFv comprises a V_{H} and a V_{L}, the V_{H} comprising V_{H} CDR1, V_{H} CDR2 and V_{H} CDR3; and the V_{L} comprising V_{L} CDR1, V_{L} CDR2 and V_{L} CDR3, wherein: (i) the V_{H} CDR1, V_{H} CDR2 and V_{H} CDR3 comprises the amino acid sequence of SEQ ID NOs: 24, 25 and 26, respectively; and (ii) the V_{L} CDR1, V_{L} CDR2 and V_{L} CDR3 comprises the amino acid sequence of SEQ ID NOs: 27, 28 and 29, respectively.

In another embodiment, the CD40-binding domain is a CD40-specific scFv comprising a V_{H} and a V_{L}, wherein: (a) the V_{H} comprises the amino acid sequence of SEQ ID NO: 22; and (b) the V_{L} comprises the amino acid sequence of SEQ ID NO: 23.

In another embodiment, the fusion protein of the invention further comprises an endoplasmic reticulum (ER) retention sequence located at the C-terminal of the antigen, with the proviso that the translocation domain comprises a PE translocation peptide (T^{PE}).

In another embodiment, the ER retention sequence comprises an amino acid sequence selected from the group consisting of SEQ ID NO: 30, 31, 32, 33 and 34.

In another embodiment, the fusion protein of the invention further comprises a CD28-activating peptide located between the CD40-binding domain and the furin and/or cathepsin L cleavage site.

In another embodiment, the CD28-activating peptide consisting of 28-53 amino acid residues in length.

In another embodiment, the CD28-activating peptide has a length of 28-53 amino acid residues, said CD28-activating peptide comprising an amino acid sequence selected from the group consisting of SEQ ID NO: 35, 36 and 37.

In another embodiment, the CD28-activating peptide has a length of 28-53 amino acid residues, said CD28-activating peptide comprising the amino acid sequence of SEQ ID NO: 35.

In another embodiment, the CD28-activating peptide comprises an amino acid sequence selected from the group consisting of SEQ ID NO: 35, 36 and 37.

In another embodiment, the CD28-activating peptide comprises an amino acid sequence that is at least 90%, 95% or 99% identical to SEQ ID NO: 35, 36 or 37.

An antigen in the fusion protein of the invention is an antigen of a pathogen or a tumor antigen.

The pathogen may be selected from the group consisting of Human Papillomavirus (HPV), Human Immunodeficiency Virus-1 (HIV-1), Influenza Virus, Dengue Virus, Hepatitis A Virus (HAV), Hepatitis B Virus (HBV), Hepatitis C Virus (HCV), Hepatitis D Virus (HDV), Hepatitis E Virus (HEV), Severe acute respiratory syndrome-associated coronavirus (SARS-COV), Severe acute respiratory syndrome coronavirus 2 (SARS-COV-2), Middle East respiratory syndrome coronavirus

(MERS-COV), Epstein-Barr virus (EBV), Zika Virus, Rabies Virus, Variola virus, Chikungunya Virus, West Nile virus, Poliovirus, Measles virus, Rubella virus, Hantavirus, Japanese encephalitis virus, Coxsackievirus, Echovirus, Enterovirus, Mumps virus, Varicella-zoster virus (VZV), Cercopithecine herpesvirus-1 (CHV-1), Yellow fever virus (YFV), Rift Valley Fever Virus, Lassa virus, Marburg virus, Ebolavirus, Norovirus, Rotavirus, Adenovirus, Sapovirus, Astrovirus, Rickettsia prowazekii, Rickettsia typhi, Orientia tsutsugamushi, Borrelia burgdorferi, Yersinia pestis, Plasmodium vivax, Plasmodium malariae, Plasmodium falciparum, Plasmodium ovale, Bacillus anthracis, Clostridium Difficile, Clostridium Botulinum, Corynebacterium diphtheriae, Salmonella enterica serovar Typhi, Salmonella enterica serovar Paratyphi A, Shiga toxin-producing E. coli (STEC), Shigella dysenteriae, Shigella flexneri, Shigella boydii, Shigella sonnei, Entamoeba histolytica, Vibrio cholerae, Mycobacterium tuberculosis, Neisseria meningitidis, Bordetella pertusis, Haemophilus influenzae type B (HiB), Clostridium tetani, Listeria monocytogenes and Streptococcus pneumoniae.

In another embodiment, the pathogen is selected from the group consisting of HPV, HIV-1,Influenza Virus, Dengue Virus, HAV, HBV, HCV, SARS-COV, SARS-COV-2. More particularly, the pathogen is selected from the group consisting of HPV, HBV, HCV and SARS-COV-2.

In another embodiment, the antigen is a pathogenic antigen selected from the group consisting of HPV₁₆ E7 protein, HPV₁₈ E7 protein, HBV X protein (HBx), HBV preS1 protein, HCV core protein (HCVcore) and SARS-COV-2 spike protein (CoV2S).

In another embodiment, said antigen comprises at least one epitope for inducing a desired immune response, preferably containing 1 to 30 epitopes, more preferably containing 1 to 15epitopes.

In another embodiment, the antigen is a pathogenic antigen comprising or consisting substantially of an amino acid sequence that is at least 70%, 80%, 90%, 95% or 99% identical to SEQ ID NO: 38, 39, 40, 41, 42 or 43.

In another embodiment, the antigen is a pathogenic antigen comprising or consisting substantially of an amino acid sequence that is at least 80% identical to SEQ ID NO: 38, 39, 40, 41,42 or 43.

In another embodiment, the antigen comprises an amino acid sequence selected from the group consisting of SEQ ID Nos: 38, 39, 40, 41, 42 and 43.

In another embodiment, the antigen is a tumor antigen. A tumor antigen is a tumor-associated antigen (TAA) or a tumor-specific antigen (TSA).

In one embodiment, the tumor or cancer is selected from the group consisting of breast cancer, colon cancer, rectal cancer, bladder cancer, endometrial cancer, kidney cancer, gastric cancer, glioblastoma, hepatocellular carcinoma, bile duct cancer, small cell lung cancer, non-small cell lung cancer (NSCLC), melanoma, ovarian cancer, cervical cancer, pancreatic cancer, prostate cancer, acute myelogenous leukemia (AML), chronic myelogenous leukemia (CML), non-Hodgkin's lymphoma, and thyroid cancer.

In another embodiment, a tumor-associated antigen is selected from the group consisting of SSX2, MAGE-A3, NY-ESO-1, iLRP, WT12-281, RNF43, CEA-NE3, AFP, ALK, Anterior gradient 2 (AGR2), BAGE proteins, B-catenin, brc-abl, BRCA1, BORIS, CA9, carbonic anhydrase IX, caspase-8, CD40, CDK4, CEA, CTLA4, cyclin-B1, CYPIB1, EGFR, EGFRvIll, ErbB2/Her2, ErbB3, ErbB4, ETV6-AML, EphA2, Fra-1, FOLRI, GAGE proteins (e.g., GAGE-1,-2), GD2, GD3, GloboH, glypican-3, GM3, gp100, HLA/B-raf, HLA/k-ras, HLA/MAGE-A3, hTERT, LMP2, MAGE proteins (e.g., MAGE-1,-2,-3,-4,-6, and-12), MART-1, mesothelin, ML-IAP, Muc1, Muc16 (CA-125), MUM1, NA17, NY-BRI, NY-BR62, NY-BR85, NY-ES01, OX40, p15, p53, PAP, PAX3, PAX5, PCTA-1, PLACI, PRLR, PRAME, PSMA (FOLHI), RAGE proteins, Ras, RGS5, Rho, SART-1, SART-3, Steap-1, Steap-2, survivin, TAG-72, TGF-β, TMPRSS2, Tn, TRP-1, TRP-2, tyrosinase, and uroplakin-3.

In another embodiment, the antigen is a tumor-associated antigen selected from the group consisting of SSX2, MAGE-A3, NY-ESO-1, iLRP, WT12-281, RNF43 and CEA-NE3.

In another embodiment, the antigen is a tumor-associated antigen comprising an amino acid sequence that is at least 70%, 80%, 90%, 95% or 99% identical to SEQ ID NO: 44, 45, 46, 47, 48, 49or 50.

In another embodiment, the antigen is a tumor-associated antigen comprising an amino acid sequence selected from the group consisting of SEQ ID NOs: 44, 45, 46, 47, 48, 49 and 50.

An antigen may be a single antigen or an antigenic fragment thereof, or a fusion antigen comprising at least two antigens fused together. For example, an antigen may be a single antigen of HPV₁₆ E7 protein or a fusion antigen comprising HPV₁₆ E7 and HPV₁₈ E7 proteins. A fusion antigen may or may not have a linker connecting different antigens.

In another embodiment, the antigen is a fusion antigen having at least one linker connecting different antigens.

In another embodiment, the antigen is a fusion antigen having a rigid linker, (EAAAAK)ₙ, connecting different antigens, wherein n is an integer from 0-12, preferably from 2-6, more preferably from 3-4. In other words, the rigid linker comprises 0 to 12 repeats, 2 to 6 repeats or 3-4 repeats of the sequence EAAAAK (SEQ ID NO: 56).

In another embodiment, the fusion protein of the invention further comprises a rigid linker between the CD40-binding domain and the furin and/or cathepsin L cleavage site. The rigid linker may be a peptide liner comprising 0 to 12 repeats of the amino acid sequence EAAAAK (SEQ ID NO: 56).

The rigid linker may be (EAAAAK)ₙ, or (SEQ ID NO: 56)ₙ, wherein n is an integer from 0-12, preferably from 2-6, more preferably from 3-4.

In another embodiment, the rigid linker comprises 2 to 6 repeats or 3-4 repeats of SEQ ID NO: 56.

In another embodiment, the fusion protein of the invention comprises, or consists substantially of, an amino acid sequence that is at least 90%, 95% or 99% identical to SEQ ID NO: 51, 52, 53, 54 or 55

Further in another embodiment, the fusion protein of the invention comprises, or consists substantially of, an amino acid sequence selected from the group consisting of SEQ ID NOs: 51, 52,53, 54 and 55.

In another aspect, the invention relates to a DNA fragment encoding a fusion protein of the invention. The invention also relates to an expressing vector comprising a DNA fragment encoding a fusion protein of the invention. The invention further relates to a pharmaceutical composition comprising a fusion protein of the invention and a pharmaceutically acceptable carrier and/or an adjuvant.

The pharmaceutical composition may be an enteral or a parenteral dosage form, suitable for transdermal, transmucosal, nasopharyngeal, pulmonary or direct injection, or for systemic (e.g., parenteral) or local (e.g., intratumor or intralesional injection) administration. Parenteral injection may be via intravenous, intraperitoneal, intramuscular, subcutaneous or intradermal routes.

Suitable adjuvants include, but not limited to, a saponin-based adjuvant or a Toll-like receptor (TLR) agonist adjuvant. A saponin-based adjuvant may be GPI-0100, Quil A or QS-21. A TLR agonist adjuvant may be Poly I: C, monophosphoryl lipid A (MPL) or CpG oligonucleotide (e.g., class A CpG: CpG1585, CpG2216 or CpG2336; class B CpG: CpG1668, CpG1826, CpG2006, CpG2007, CpG BW006 or CpG D-SL01; class C CpG: CpG2395, CpG M362 or CpG D-SL03). In one embodiment, the adjuvant is a CpG oligonucleotide.

The pharmaceutical composition may also be administered orally, e.g., in the form of tablets, coated tablets, dragées, hard and soft gelatine capsules.

The dosage of the fusion protein may vary, depending on the disease to be controlled, the age and the individual condition of the patient and the mode of administration. The dosage may be fitted to individual requirements in each particular case so as to obtain a therapeutically effective amount of the fusion protein of the invention to achieve a desired therapeutic response.

For adult patients, a single dosage of about 0.1 to 50 mg, especially about 0.1 to 5 mg, comes into consideration. Depending on severity of the disease and the precise pharmacokinetic profile, the fusion protein may be administered with one dosage unit per week, bi-week or month, and totally give 1 to 6 dosage units per cycle to satisfy such treatment.

In another aspect, the invention relates to use of the fusion protein or the pharmaceutical composition of the invention in the manufacture of a medicament for eliciting an antigen-specific T cell immune response, protecting against and/or treating an infectious disease or a tumor in a subject in need thereof.

Abbreviations: Rap1. Ras-proximate-I or Ras-related protein 1: CD40, Cluster of differentiation 40; CDR, Complementarity-determining region.

### EXAMPLES

### Animal Tumor Model

An HPV₁₆ E6-and E7-expressing tumor cell line from lung epithelial cells of C57BL/6 mice was used to establish a mouse HPV₁₆ tumor model for in vivo efficacy assays in the examples 6-8. The tumor cells were grown in RPMI 1640 medium containing FBS (10%) and penicillin/streptomycin/Amphotericin B (50 units/mL) at 37° C., 5% CO2.

### SEQ ID NOs. and Components

TABLE 1

| SEQ | | |
|---|---|---|
| ID | | Length |
| No. | Component name or sequence (N → C) | (aa) |
| 1 | Cleavable linker 1 | 4 |
| | RX¹X²R, wherein X¹ and X² are any amino acid | |
| | residue. | |
| 2 | Cleavable linker 2 | 6 |
| | RX¹RX²X³R, wherein X¹ and X² are any amino acid | |
| | residue, and X³ is K, F or R. | |
| 3 | Rigid linker 1 (EAAAAK)₃ | 18 |
| 4 | Full length PE | 613 |
| 5 | PE translocation peptide (PE₂₈₀₋₃₀₅, minimal) | 26 |
| 6 | PE translocation peptide (PE₂₈₀₋₃₁₃) | 34 |
| 7 | PE translocation peptide (PE₂₆₈₋₃₁₃) | 46 |
| 8 | PE translocation peptide (PE₂₅₃₋₃₁₃) | 61 |
| 9 | PE translocation peptide (PE₂₅₃₋₃₆₄) | 112 |
| 10 | Full length Shiga toxin (Stx) subunit A | 293 |
| 11 | Full length Shiga-like toxin I (Slt-I) subunit A | 293 |
| 12 | Stx translocation peptide (Stx₂₄₀₋₂₄₇, minimal) | 8 |
| 13 | Stx translocationpsptide (Stx₂₄₀₋₂₅₁) | 12 |
| 14 | Stx translocation peptide (Stx₂₁₁₋₂₄₇) | 37 |
| 15 | Stx translocation peptide (Stx₂₁₁₋₂₅₁) | 41 |
| 16 | Stx translocation peptide (Stx₁₆₈₋₂₅₁) | 84 |
| 17 | Full length CD40 ligand (CD40L₁₋₂₆₁) | 261 |
| 18 | Truncated CD40 ligand (CD40L₄₇₋₂₆₁) | 215 |
| 19 | Truncated CD40 ligand (CD40L₁₀₈₋₂₆₁, also referred | 154 |
| | to as 18sCD40L) | |
| 20 | Anti-CD40 scFv (V_{H}-L-V_{L}) | 246 |
| 21 | Anti-CD40 scFv (V_{L}-L-V_{H}) | 246 |
| 22 | V_{H} of the anti-CD40 scFv | 119 |
| 23 | V_{L} of the anti-CD40 scFv | 112 |
| 24 | V_{H} CDR1 GFTFSTYGMH | 10 |
| 25 | V_{H} CDR2 GKGLEWLSYISGGSSYIFYADSVRGR | 26 |
| 26 | V_{H} CDR3 CARILRGGSGMDL | 13 |
| 27 | V_{L} CDR1 CTGSSSNIGAGYNVY | 15 |
| 28 | V_{L} CDR2 GNINRPS | 7 |
| 29 | V_{L} CDR3 CAAWDKSISGLV | 12 |
| 30 | ER retention sequence KDEL | 4 |
| 31 | ER retention sequence KKDLRDELKDEL | 12 |
| 32 | ER retention sequence KKDELRDELKDEL | 13 |
| 33 | ER retention sequence KKDELRVELKDEL | 13 |
| 34 | ER retention sequence KDELKDELKDEL | 12 |
| 35 | CD28 consensus sequence | 28 |
| | T¹D²I³Y⁴F⁵C⁶K⁷X⁸E⁹X¹⁰X¹¹Y¹²P¹³P¹⁴P¹⁵Y¹⁶X¹⁷ | |
| | D¹⁸N¹⁹E²⁰K²¹S²²N²³G²⁴T²⁵I²⁶I²⁷H²⁸, wherein | |
| | X⁸ is I or L, X¹⁰ is V, F or A, X¹¹ is M or L, X¹⁷ is | |
| | L or I. | |
| 36 | CD28-activating peptide (minimal) | 28 |
| 37 | CD28-activating peptide | 53 |
| 38 | Antigen HPV₁₆ E7 protein | 98 |
| 39 | Antigen HPV₁₈ E7 protein | 104 |
| 40 | Antigen HBV X protein (HBx; full length) | 154 |
| 41 | Antigen HBV preS1 protein | 108 |
| 42 | Antigen HCV core protein (full length) | 190 |
| 43 | Antigen SARS-CoV-2 spike protein | 1273 |
| 44 | Antigen SSX2 | 187 |
| 45 | Antigen MAGE-A3 | 314 |
| 46 | Antigen NY-ESO-1 | 180 |
| 47 | Antigen iLRP | 296 |
| 48 | Antigen WT12-281 | 279 |
| 49 | Antigen RNF43 | 406 |
| 50 | Antigen CEA-NE3 | 284 |
| 51 | Fusion protein CD40L₄₇₋₂₆₁-T^{PE}-E7 | 528 |
| 52 | Fusion protein 18sCD40L-T^{PE}-E7 | 467 |
| 53 | Fusion protein E7-T^{Stx}-CD40L₄₇₋₂₆₁ | 535 |
| 54 | Fusion protein E7-T^{Stx}-18sCD40L | 474 |
| 55 | Fusion protein HBx-preS1-T^{Stx}-18sCD40L | 541 |
| 56 | Rigid linker EAAAAK | 6 |

Flow cytometry. Splenocytes were stimulated with an antigenic stimulator for 2 hours at 37° C., followed by treating with 50 μg/mL of Brefeldin A and Monensin at 37° C. for 2 hours. The cells were harvested, washed with PBS containing 0.5% BSA, and stained with APC/Cy7-conjugated anti-CD3 antibody, PerCP/Cy5.5-conjugated anti-CD4 antibody, FITC-conjugated anti-CD8 antibody, PE-conjugated anti-CD44 antibody and APC-conjugated anti-CD62L antibody simultaneously. After wash, the cells were permeabilized, fixed and intracellularly stained with PE-conjugated anti-IFN-γ antibody, PE/Cy7-conjugated anti-IL-2 antibody and eFluor450-conjugated anti-TNF-a antibody simultaneously. The intracellular cytokine characterization (IFN-γ, IL-2 or TNF-a) of splenocytes with CD8+ or CD4+ memory T cell phenotypes (CD3+CD44^{hi}CD62L10) were further analyzed by Gallios flow cytometer and Kaluza software.

Enzyme-linked immunospot (ELISpot) assay. Splenocytes were seeded in triplicate in a pretreated murine IFN-γ capturing 96-well plate (CTL IMMUNOSPOT®) at a cell density of 2×10⁵ cells/well in the presence or absence of an antigenic stimulator. The cells were discarded after 24 hours of incubation at 37° C. After wash, the captured IFN-γ was detected by biotin-conjugated anti-murine IFN-γ antibody at room temperature for 2 hours and the IFN-γ-immunospots were developed according to the manufacturer's instructions. The scanning and counting of IFN-γ-immunospots was performed by IMMUNOSPOT® S5 Micro analyzer (CTL).

Indirect enzyme-linked immunosorbent assay (ELISA). Collected whole blood samples were left undisturbed at 4° C. for 30-60 minutes followed by centrifugation at 5,000g for 10 minutes to pellet the clot. The serum samples were stored at-20° C. The purified coating protein for antigen-specific antibody binding was diluted in guanidine coating buffer (2 M guanidine hydrochloride, 500mM Na₂HPO₄, 25 mM citrate, pH 4.0-4.4) and distributed into 96-well plate at 1 μg/well. After overnight incubation at 4° C., the 96-well plate was blocked with 1% BSA in PBS at 37° C. for 1 hour. The serum samples were thawed, and subsequently 10-fold serial diluted in PBS with 1% BSA. The coated protein was incubated with 100 μl of 1000-fold diluted serum sample at 37°° C. for 2 hours. After 4 times washing with phosphate buffered saline TWEEN®-20 (PBST), the antigen-specific antibodies were detected by horseradish peroxidase (HRP)-conjugated goat anti-mouse IgG (at a dilution of 1:10,000, Cat #31430, Thermo Fisher Science) at 37° C. for 30 minutes. Following 4 times of washing with PBST, the HRP-mediated color development was catalyzed in the presence of 100uL of TMB substrate and quenched by 100 μL of 1 N HCl. The relative titers of antigen-specific antibody in the serum samples were determined by the absorbance at 450 nm.

Statistical analysis. The significance of all comparisons was calculated by using t-test, and results considered significant when p<0.05.

### Example 1

### Constructions of Expression Vectors for CD40L₄₇₂₆₁-T^{PE}-E7 and 18sCD40L-T^{PE}-E7

FIGS. 5A-E illustrates various embodiments of the fusion protein according to the invention.

The fusion protein CD40L₄₇₋₂₆₁-T^{PE}-E7 (SEQ ID NO: 51; FIG. 5A) comprises (a) a truncated CD40 ligand CD40L₄₇₋₂₆₁ (SEQ ID NO: 18); (b) a cleavable linker comprising (EAAAAK)₃ (SEQ ID NO: 3) and RX¹RX²X³R (SEQ ID NO: 2) (wherein X¹ is A, X² is Y, X³ is K); (c) a PE translocation peptide of SEQ ID NO: 5 (PE₂₈₀₋₃₀₅); and (d) a fusion antigen HPV_{16 18} E7, comprising a HPV₁₆ E7 protein of SEQ ID NO: 38 and a HPV₁₈ E7 protein of SEQ ID NO: 39.

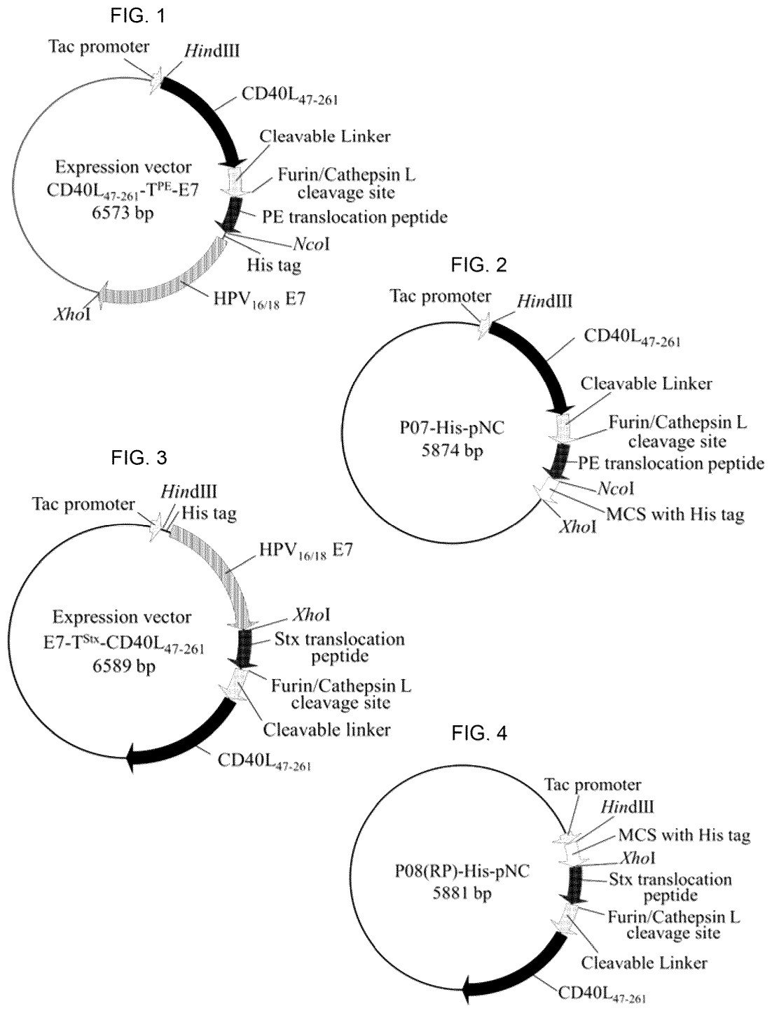

An expression vector for CD40L₄₇₋₂₆₁-T^{PE}-E7 (FIG. 1) is constructed as follows: A DNA fragment encoding ^{HindIII}CD40L-Linker-PE^{NcoI, XhoI, SalI}, comprising the CD40L_{47-261,} the cleavable linker and the PE translocation peptide (PE₂₈₀₋₃₀₅), was PCR synthesized, digested by HindIII/SalI and then ligated into the plasmid pTAC-MAT-Tag-2 having HindIII/XhoI cutting sites to obtain the plasmid P07-His-pNC (FIG. 2). Then, a DNA fragment encoding a fusion antigen HPV_{16/18} E7 carrying a His tag was inserted into the plasmid PO7-His-pNC (FIG. 2) via restriction enzymes NcoI/XhoI to generate the expression vector for the fusion protein CD40L₄₇₋₂₆₁-T^{PE}-E7 (FIG. 1).

A cleavable linker allows furin and/or cathepsin L protease to cut the fusion protein for releasing the T^{PE}-E7 fragment from the fusion protein.

Applying a similar method as described above, any other antigen(s) of interest may be used to replace E7 and be inserted into the plasmid of FIG. 2 to generate an expression vector similar to the plasmid of FIG. 1 for a fusion protein comprising the antigen of interest according to the invention.

An expression vector for the fusion protein 18sCD40L-T^{PE}-E7 (SEQ ID NO: 52; FIG. 5B) was constructed using a similar method described above, in which the truncated CD40 ligand: CD40L₄₇₋₂₆₁ (SEQ ID NO: 18) was replaced by 18sCD40L, another truncated CD40 ligand: CD40L₁₀₈₂₆₁ (SEQ ID NO: 19).

### Example 2

### Construction of Expression Vectors for E7-T^{Stx}-CD40L₄₇₋₂₆₁ and E7-T^{Stx}-18sCD40L

The fusion protein E7-T^{Stx}-CD40L₄₇₋₂₆₁ (SEQ ID NO: 53; FIG. 5C) comprises (a) a fusion antigen HPV_{16/18} E7 (comprising HPV₁₆ E7 protein (SEQ ID NO: 38) and HPV₁₈ E7 protein (SEQ ID NO: 39)), (b) a Stx translocation peptide of SEQ ID NO: 14 (Stx₂₁₁₋₂₄₇), (c) a cleavable linker comprising RX¹X²R of SEQ ID NO: 1 (wherein X¹ is V, X² is A) and (EAAAAK)₃ of SEQ ID NO: 3, and (d) a truncated CD40 ligand of SEQ ID NO: 18 (CD40L₄₇₋₂₆₁).

An expression vector for E7-T^{Stx}-CD40L₄₇₋₂₆₁ (FIG. 3) is constructed as follows:

A DNA fragment encoding ^{HindIII, XhoI}Stx-Linker-CD40L^{SalI}, comprising the Stx translocation peptide (Stx₂₁₁₋₂₄₇), the cleavable linker and the CD40L₄₇₋₂₆₁, was PCR synthesized, digested by HindIII/Sa/I, then ligated into plasmid pTAC-MAT-Tag-2 backbone having HindIII/Xhol cutting sites to obtain the plasmid P08 (RP)-His-pNC (FIG. 4). Then, another DNA fragment encoding a fusion antigen HPV_{16/18} E7 carrying a His tag was inserted into the plasmid P08 (RP)-His-pNC (FIG. 4) via restriction enzymes HindIII/Xhol to generate the expression vector E7-T^{Stx}-CD40L₄₇₋₂₆₁ (FIG. 3).

The cleavable linker is vital for the fusion protein of the invention because it allows the fusion protein to be cut by furin and/or cathepsin L protease so as to release the E7-T^{Stx} fragment from the fusion protein. For example, see FIG. 5C.

Applying a similar method as described above, any other antigen(s) of interest from various pathogens or cancer may replace E7 and be inserted into the plasmid of FIG. 4 to generate an expression vector similar to the plasmid of FIG. 3 for a fusion protein comprising the antigen of interest according to the invention.

Using a similar method described above, an expression vector for the fusion protein E7-T^{Stx}-18sCD40L (SEQ ID NO: 54; FIG. 5D) was constructed, in which the truncated CD40 ligand: CD40L₄₇₋₂₆₁ (SEQ ID NO: 18) was replaced by 18sCD40L, another truncated CD40 ligand: CD40L₁₀₈₋₂₆₁ (SEQ ID NO: 19).

For a comparison purpose, we have constructed the fusion protein RAP1-CD28_{conv}PEₜ-E7-K3 (referred to as “RAP1-E7” in the present application), which was almost identical to the prior construct disclosed in U.S. Pat. No. 9,481,714 B2, Example 1. The RAP1-CD28_{conv}PEₜ-E7-K3 (referred as “RAP1-E7” in the application) comprises a RAPI domain III, a CD28 sequence, a linker, a PE translocation domain II (PE₂₆₈₋₃₁₃), an antigen E7 protein and an endoplasmic reticulum retention sequence. The antigen E7 protein used here is a fusion antigen HPV_{16/18} E7, which comprises a HPV₁₆ E7 protein (SEQ ID NO: 38) and HPV₁₈ E7 protein (SEQ ID NO: 39), while the antigen E7 protein used in the prior art is HPV₁₆ E7 protein.

### Example 3

### Construction of Expression Vectors for HBx-preS1-T^{Stx}-18sCD40L

The fusion protein HBx-preS1-T^{Stx}-18sCD40L (SEQ ID NO: 55; FIG. 5E) comprises (a) a fusion antigen HBx-preSI comprising a HBx protein of SEQ ID NO: 40 and a HBV preS1 protein of SEQ ID No.41, (b) a Stx translocation peptide of SEQ ID NO: 14 (Stx₂₁₁₋₂₄₇), (c) a cleavable linker comprising RX¹X²R of SEQ ID NO: 1 (wherein X¹ is V, X² is A) and (EAAAAK)₃ of SEQ ID NO: 3,and (d) a truncated CD40 ligand of SEQ ID NO: 19 (18sCD40L).

Using a similar method described in Example 2, an expression vector for the fusion protein HBx-preS1-T^{Stx}-18sCD40L was constructed, in which the truncated CD40 ligand used was 18sCD40L and the antigen used was the fusion antigen HBx-preSI as described above.

### Example 4

### Protein Expression

E. coli BL21 cells harboring the protein expression vector CD40L₄₇₋₂₆₁-T^{PE}-E7 were inoculated in ZY media (10 g/L tryptone and 5 g/L yeast extract) containing a selected antibiotic at an appropriate concentration at 37° C. When the culture reached an early log phase, (OD₆₀₀=2 to 5), the expression of fusion protein was induced by isopropyl-1-thio-μ-D-galactopyranoside (IPTG) (0.5 to 2 mM). Cells were harvested after 4 hours of IPTG induction and disrupted by sonication. The inclusion bodies were isolated and solubilized in solubilization buffer (6 M guanidine hydrochloride, 20 mM potassium phosphate, 500 mM NaCl, 20 mM imidazole, 1 mM DTT, pH 7.4) for the recovery of overexpressed fusion protein. After purification, the refolding of the fusion protein was performed by dialysis against 20-to 50-fold volume of dialysis buffer (10 mM PBS) at 4° C. overnight. The refolded fusion proteins were subject to SDS-PAGE analyses under reduced (with dithiothreitol; +DTT) and non-reduced (without dithiothreitol; −DTT) conditions to evaluate whether they were properly refolded.

The following fusion proteins were also expressed and refolded by using a similar method as described above: (1) 18sCD40L-T^{PE}-E7; (2) E7-T^{Stx}-CD40L₄₇₋₂₆₁; (3) E7-T^{Stx}-18sCD40L; (4) RAP1-E7; (5) CD40L₄₇₋₂₆₁-T^{PE}-HBx-preS1; (6) 18sCD40L-T^{PE}-HBx-preS1; (7) HBx-preS1-T^{Stx}-CD40L47-261;(8) HBx-preS1-T^{Stx}-18sCD40L; (9) CD40L₄₇₋₂₆₁-T^{PE}-HCVcore; (10) 18sCD40L-T^{PE}-HCVcore; (11) HCVcore-T^{Stx}-CD40L47-261; (12) HCVcore-TStx-18sCD40L; (13) CD40L₄₇₋₂₆₁-T^{PE}-CoV2S; (14) 18sCD40L-T^{PE}-CoV2S; (15) CoV2S-TStx-CD40L47-261; (16) CoV2S-TStx-18sCD40L; (17) CD40L₄₇₋₂₆₁-TPE-SSX2; (18) 18sCD40L-T^{PE}-SSX2; (19) SSX2-T^{Stx}-CD40L₄₇₋₂₆₁; (20) SSX2-T^{Stx}-18sCD40L.

The fusion proteins CD40L₄₇₋₂₆₁-TPF-E7, 18sCD40L-T^{PE}-E7, E7-T^{Stx}-18sCD40L, RAP1-E7 and HBx-preS1-T^{Stx}-18sCD40L were further subjected to an immunogenicity analysis or an efficacy analysis in the following experiments.

Example 5

### Immunogenicity Analysis of Fusion Proteins

Female C57BL/6NCrlBltw mice (5 to 6-weck-old) were randomly divided into 5 groups (n=5): (A) placebo (i.c., PBS); (B) fusion protein CD40L₄₇₋₂₆₁-T^{PE}-E7 (100 μg); (C) fusion protein 18sCD40L-T^{PE}-E7 (100 ug); (D) fusion protein E7-T^{Stx}-18sCD40L (100 μg); and (E) fusion protein RAPI-E7 (100 μg). The fusion proteins were dialyzed into PBS. CpG1826 (50 μg) was used as an adjuvant to animal groups B to E. Each group received three immunizations subcutaneously (s.c.) at 7 days interval from day 0. Blood samples were collected on day 0, 7 and 14. On day 21, the blood samples were harvested and the splenocytes were resuspended in RPMI 1640 medium containing FBS (10%) and PSA.

The splenocytes were used to analyze intracellular cytokine induction (IFN-γ, IL-2 and TNF-α) in the CD8⁺ and CD4⁺ memory T cells in the presence or absence of antigen stimulation. Briefly, splenocytes from each animal group were treated with or without antigen E7 protein (2 μg/mL of HPV₁₆ E7 peptide pool) and then analyzed by flow cytometry. The degree or the level of the intracellular cytokine induction in each mouse group was presented as relative cytokine induction, which was obtained by normalizing the frequency of cytokine⁺/CD8⁺ and cytokine⁺/CD4⁺splenocytes in the presence of the stimulating antigen E7 to that of the unstimulated (untreated) control.

The splenocytes were also used to analyze the frequency of IFN-γ-secreting splenocytes in the presence or absence of antigen stimulation (2 μg/mL of HPV₁₆ E7 peptide pool) by using Enzyme-linked immunospot (ELISpot) assay. The results were presented as IFN-γ⁺immunospots per million splenocytes.

The blood samples were used to analyze the level of serum HPV₁₆ E7-specific and HPV₁₈ E7-specific antibody by using ELISA, in which the purified HPV₁₆ E7 and HPV₁₈ E7 recombinant proteins were used as coating proteins, respectively.

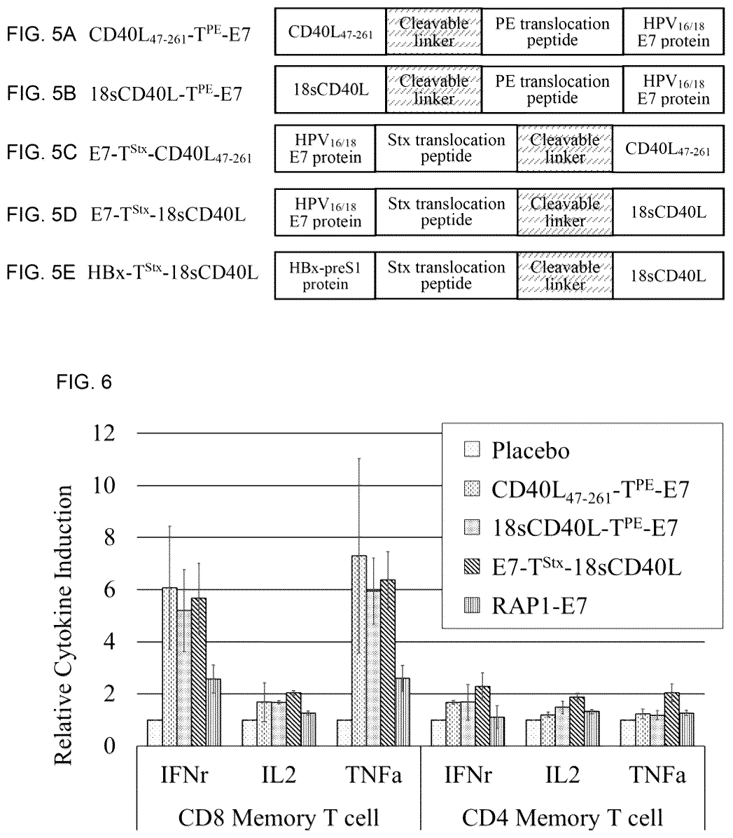

FIG. 6 shows cytokine induction results after antigen stimulation of the splenocytes with HPV₁₆ E7 peptide pool. The relative cytokine induction of IFN-γ and TNF-α, but not IL2, in CD8⁺memory T cells from the animals immunized with the fusion protein CD40L₄₇₋₂₆₁-T^{PE}-E7, 18sCD40L-T^{PE}-E7or E7-T^{Stx}-18sCD40L (Groups B-D) all significantly increased as compared to that from the RAP1-E7-treated group (Group E) or the placebo group (Group A). The relative cytokine induction of IL-2 in CD8 memory T cells, and the cytokines IFN-γ, IL-2 or TNF-α in CD4⁺memory T cells in the animal groups B-E slightly increased, however, showed no significant difference as compared to placebo group (Group A).

Nonetheless, it can be concluded that the fusion protein of the invention is superior to the prior art fusion protein in inducing the expression of IFN-γ and TNF-α in CD8 memory T cells in response to the stimulation of the antigen HPV₁₆ E7.

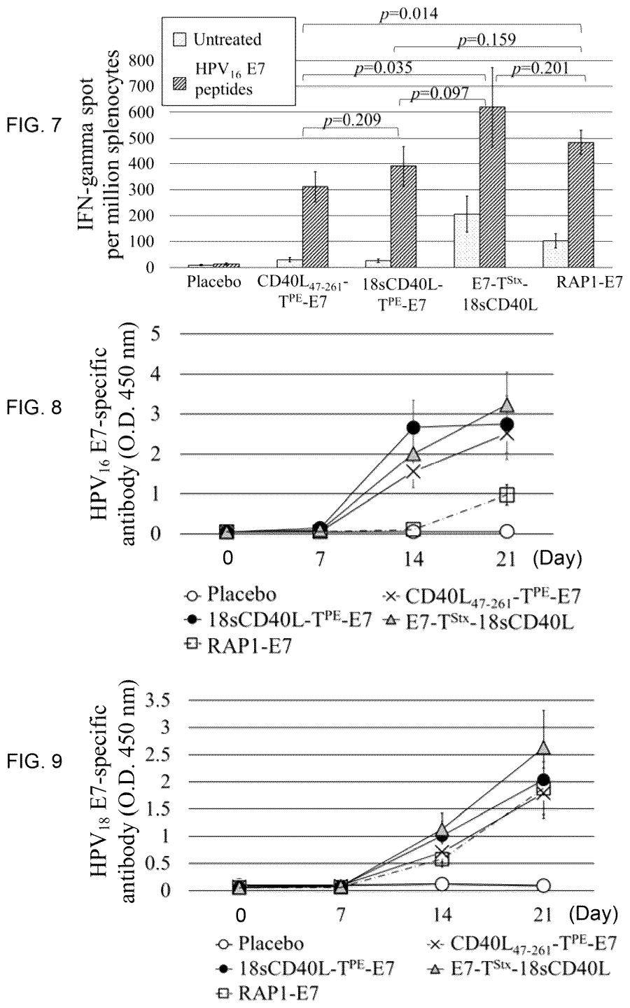

FIG. 7 shows IFN-γ⁺ immunospots in the splenocytes stimulated with the HPV₁₆ E7 peptide pool in vitro. The frequency of IFN-γ-secreting splenocytes from the animal groups immunized with CD40L₄₇₋₂₆₁-T^{PE}-E7, 18sCD40L-T^{PE}-E7, E7-T^{Stx}-18sCD40L and RAP1-E7 (Groups B-E), respectively, significantly increased as compared to the placebo group. Particularly, E7-T^{Stx}-18sCD40L induced significantly higher frequency of IFN-γ-secreting cells than CD40L₄₇₋₂₆₁-T^{PE}-E7 (p=0.035).

The results indicate that the fusion protein of the invention can significantly increase IFN-γ-secreting T cell population upon or after stimulation with the antigenic HPV₁₆ E7 peptide pool.

FIG. 8 shows the serum HPV₁₆ E7-specific antibody levels in the animals immunized with various fusion proteins on day 0, 7 and 14. The HPV₁₆ E7-specific antibody level started to increase after the second vaccination on day 7, and further rose after the third vaccination on day 14 in animals vaccinated with CD40L₄₇₋₂₆₁-T^{PE}-E7, 18sCD40L-T^{PE}-E7 or E7-T^{Stx}-18sCD40L (Groups B-D respectively). On day 21, the serum HPV₁₆ E7-specific antibody levels in Groups B-D animals were higher than the placebo and the animal group vaccinated with RAP1-E7 (i.e., RAP1-CD28convPEt-E7-K3).

The fusion protein RAP1-E7 (RAP1-CD28convPEt-E7-K3) failed to elicit HPV 16 E7-specific antibody level after two vaccinations (on day 0 and 7). It started to induce HPV₁₆ E7-specific antibody after the third vaccination on day 14, and the serum antibody level was only modest on day 21 as compared to Groups B-D. In contrast, the fusion protein of the invention elicited serum HPV16 E7-specific antibody level after two shots of the vaccine on day 0 and 7.

A similar effect in inducing HBx-specific antibody was also observed when animals were vaccinated with RAP1-CD28convPEt-HBx-K3 (referred to as “RAP1-HBx”), using the same regimen and immunization schedule described above. The fusion protein RAP1-HBx was generated by using HBx antigen to replace the E7 antigen in the RAP1-E7 (RAP1-CD28convPEt-E7-K3). The fusion protein RAP1-HBx induced serum HBx-specific antibody level after the third vaccination on day 14, and the serum antibody level on day 21 was only modest as compared to animals vaccinated with the fusion protein of the invention (data not shown).

FIG. 9 shows the serum HPV₁₈ E7-specific antibody level in the animals immunized with various fusion proteins on day 0, 7 and 14. The fusion proteins CD40L₄₇₋₂₆₁-T^{PE}-E7, 18sCD40L-T^{PE}-E7 and E7-T^{Stx}-18sCD40L (Groups B-D, respectively) significantly increased the serum HPV₁₈ E7-specific antibody level as compared to the placebo group.

Thus, the fusion protein of the invention is effective in inducing antigen-specific antibodies and the antibody induction occurs after twice vaccinations.

In summary, the fusion protein of the invention can induce antigen-specific T cell response, increase the expression of proinflammatory cytokines, e.g., IFN-γ and TNF-α, and generate antigen-specific antibody response.

### Example 6

### In Vivo Efficacy Assay of Fusion Proteins

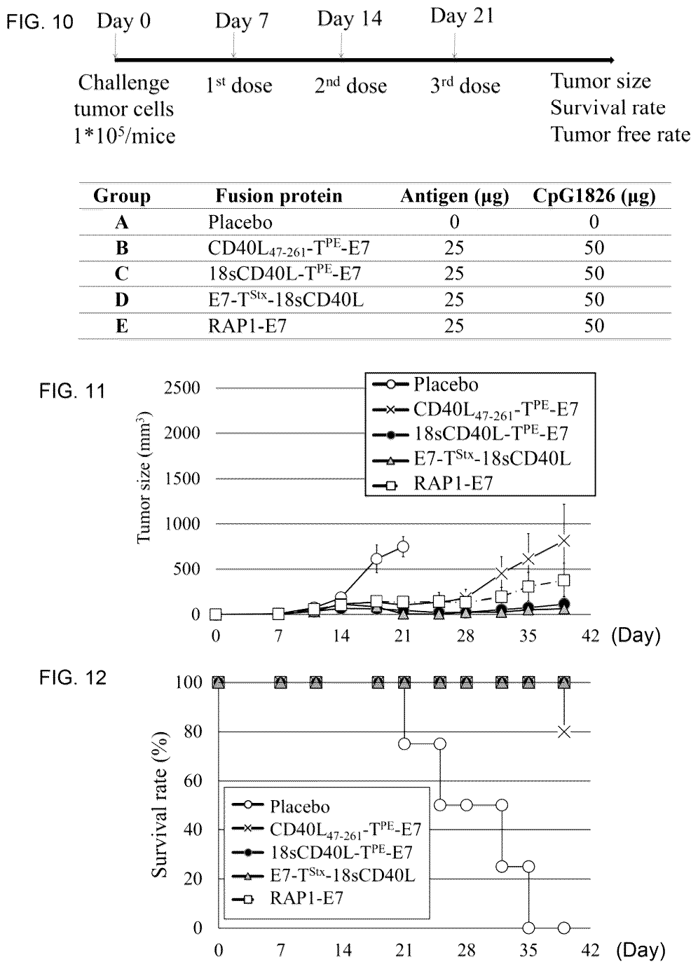

Female C57BL/6NCrlBltw mice (5 to 6-week-old) were randomly divided into 5 groups and treated with PBS (Group A, placebo, n=4); or one of the following fusion proteins: Group B, CD40L₄₇₋₂₆₁-T^{PE}-E7 (25 μg; n=5); Group C, 18sCD40L-T^{PE}-E7 (25 μg; n=4); Group D, E7-T^{Stx}-18sCD40L (25 μg; n=5); and Group E, RAP1-E7 (25 μg; n=5). The fusion proteins were dissolved in PBS and CpG1826 (50 μg) was used as an adjuvant in vaccinating animals in Groups B to E. FIG. 10 shows an immunization schedule, fusion proteins and the dosages.

To challenge mice, tumor cells (1×10⁵ in 0.1 mL) were injected s.c. into the left flank of each mouse on day 0. Three immunizations were s.c. given on day 7, 14 and 21. The tumor size was determined twice a week by multiplication of caliper measurements based on the modified ellipsoidal formula: Tumor volume=1/2 (length×width²). The survival rate and tumor free rate were calculated. Mice with tumor length over 2 cm were considered dead and mice without measurable or palpable tumor masses were considered tumor-free.

The inoculated tumor developed rapidly in the placebo group, in which two animals died on day 25 and thus the data for the placebo group were shown only until day 21 (FIG. 11). The tumor masses in the Groups C and D animals (immunized with 18sCD40L-T^{PE}-E7 and E7-T^{Stx}-18sCD40L, respectively) were almost completely suppressed at least during the entire experimental period (last day is Day 39). The tumors in Group B and E animals (immunized with CD40L₄₇₋₂₆₁-T^{PE}-E7 and RAP1-E7, respectively) were initially well controlled, however, gradually grew after ceasing immunization.

The results indicate that the fusion protein of the invention, particularly the fusion proteins 18sCD40L-T^{PE}-E7 and E7-T^{Stx}-18sCD40L, can effectively suppress tumor growth.

The survival rate in the animal groups B-E (immunized with CD40L₄₇₋₂₆₁-T^{PE}-E7, 18sCD40L-T^{PE}-E7, E7-T^{Stx}-18sCD40L and RAP1-E7, respectively) remained 100% on day 35 as compared to the placebo group, which declined to 0% on day 35 (FIG. 12).

The results indicate that the fusion protein of the invention can effectively maintain the survival rate in the animal tumor model.

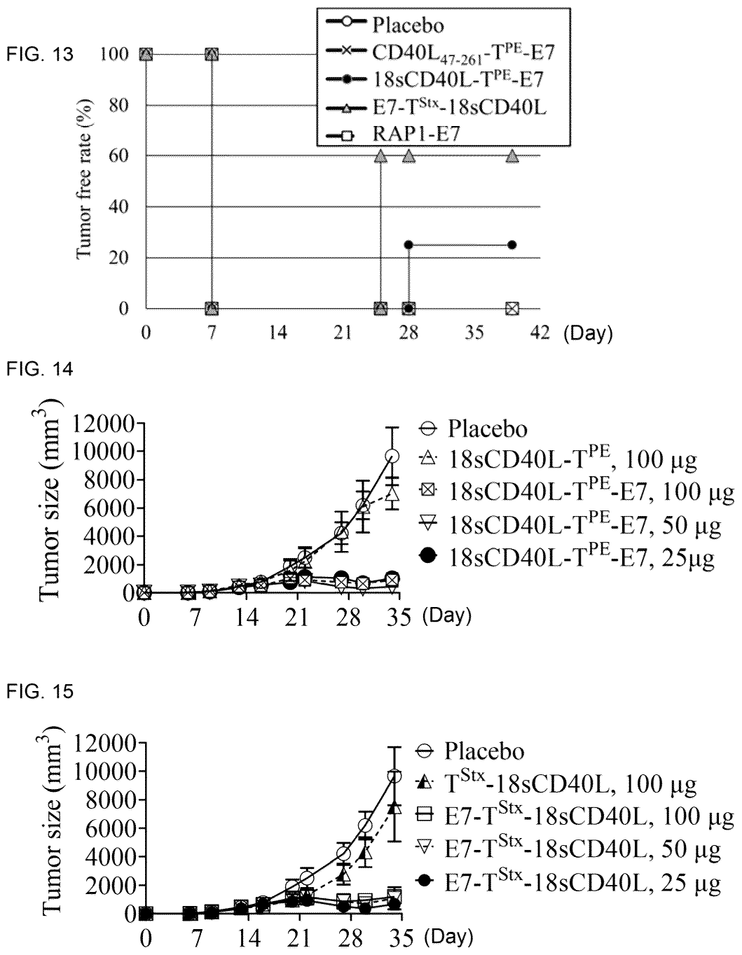

No tumor-free animals could be found in Groups A, B and E animals during the entire experimental period (day 39) (FIG. 13). One animal (25%) in group C and three animals (60%) in group D (immunized with 18sCD40L-T^{PE}-E7 and E7-T^{Stx}-18sCD40L, respectively) were found surviving without measurable or palpable tumors. Notably, in those tumor-free mice the tumor masses were all eliminated soon after completion of three times immunizations with 18sCD40L-T^{PE}-E7 or E7-TStx-18sCD40L.

The results indicated that the fusion proteins of the invention are more potent than the prior art fusion protein RAP1-E7 in increasing tumor free rate in animals having tumors.

### Example 7

### In Vivo Efficacy Analysis on Different Doses of 18sCD40L-T^{PE}-E7

Female C57BL/6NCrlBltw mice (4 to 6-week-old) were randomly divided into 5 groups (n=5per group): (A) placebo (PBS); (B) 18sCD40L-T^{PE} (100 μg; without the fusion antigen E7); (C) 18sCD40L-T^{PE}-E7 (100 ug); (D) 18sCD40L-T^{PE}-E7 (50 μg); (E) 18sCD40L-T^{PE}-E7 (25 μg). The fusion proteins were dissolved in PBS and CpG1826 (50 μg) used as an adjuvant in Groups B to E. Tumor cells (1×10⁶ in 0.1 mL) were injected s.c. into the left flank of each mouse on day 0. Two weeks after the challenge, tumor mice were vaccinated three times s.c. on day 14, 21 and 28.

The tumor volume was determined. All the dosages (25 μg, 50 μg or 100 μg) of the fusion protein 18sCD40L-TPE-E7 showed potent effects in suppressing tumor growth. The inhibition of the tumor size by the fusion protein was seen after the first shot on day 14, sustained through the entire experimental period until the last day of observation on day 34. The placebo and 18sCD40L-T^{PE}, both lacking the antigen E7, had no effect in suppressing tumor growth (FIG. 14).

### Example 8

### In Vivo Efficacy Analysis on different doses of E7-T^{Stx}-18sCD40L

Mice were grouped, challenged with tumor cells, and dosed on day 14, 21, 28 with the fusion protein, and the tumor size measured using a method similar to Example 7, except that Groups B-E mice were vaccinated with (B) T^{Stx}-18sCD40L (100 μg; without the fusion antigen E7); (C) E7-T^{Stx}-18sCD40L (100 μg); (D) E7-T^{Stx}-18sCD40L (50 μg); and (E) E7-TStx-18sCD40L (25 μg), respectively. All the dosages (25 μg, 50 μg or 100 μg) of the fusion protein E7-T^{Stx}-18sCD40L showed potent effects in suppressing tumor growth (FIG. 15). The inhibition of the tumor size by the fusion protein was seen after the first shot, sustained through the entire experimental period until the last day of observation on day 34. The placebo and T^{Stx}-18sCD40L, both lacking the antigen E7, had no effect in suppressing tumor growth (FIG. 15).

Thus, the fusion protein of the invention has potent effects in suppressing tumor growth with outstanding therapeutic efficacy.

### Example 9

### Number of Vaccine Doses: Immunogenicity Analysis of HBx-preS1-T^{Stx}-18sCD40L

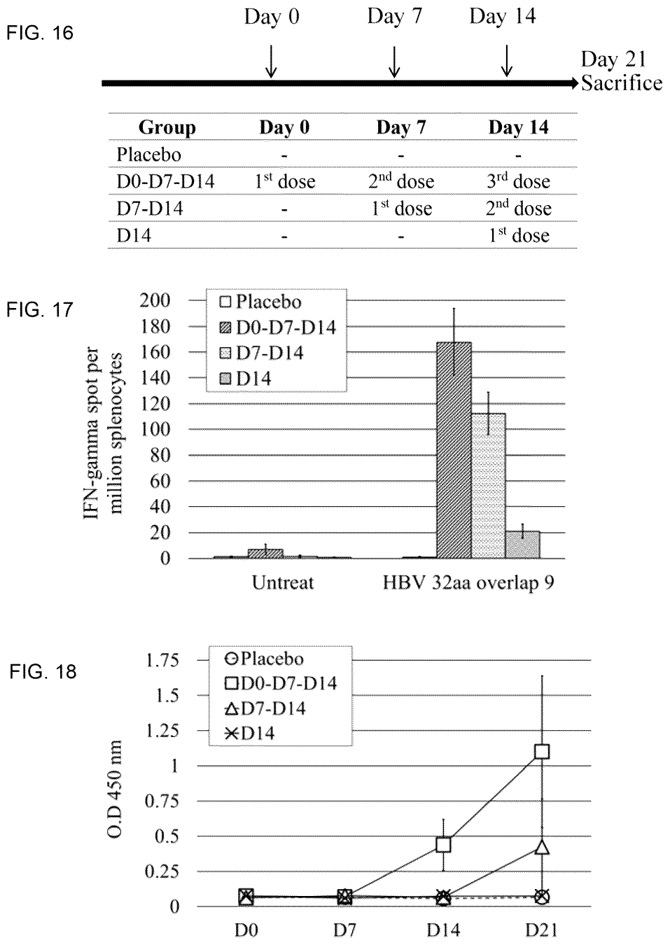

FIG. 16 shows each animal group's dosing schedule. C57BL/6JNarl female mice (5 weeks old) were randomly divided into four groups (n=5 per group): (1) placebo group; (2) DO-D7-D14 group (three doses, vaccinated on days 0, 7, 14); (3) D7-D14 (two doses, vaccinated on days 7, 14); and (4) D14 group (one dose, vaccinated on day 14). The placebo group received PBS via s.c. on Day 0, 7 and 14. Mice in other groups received HBx-preS1-T^{Stx}-18sCD40L (100 ug) adjuvanted with CpG1826 ODN (50ug) via s.c. according to the dosing schedule in FIG. 16. Blood samples were collected on day 0, 7, 14 and 21. On day 21, the animals were sacrificed, splenocytes harvested and cultured. The frequency of IFN-γ-secreting splenocytes in the presence and absence of an antigenic stimulator (a HBx-specific peptide pool, i.e., HBV 32aa overlap 9 peptide) was analyzed by ELISpot assay, respectively. The levels of serum HBx-specific antibodies were assayed by ELISA, in which purified HBx recombinant proteins were used as coating proteins.

FIG. 17 shows the IFN-γ⁺ immunospots in the splenocytes stimulated with the HBV 32aa overlap 9 peptide pool in vitro in each animal group. The results indicate that the splenocytes from animal groups immunized with three doses, two doses and one dose (groups DO-D7-D14, D7-D14and D14, respectively) all show a significant increase in the frequency of IFN-γ-secreting splenocytes as compared to the placebo. The frequency of IFN-γ-secreting splenocytes was positively correlated with the number of immunizations. The group DO-D7-D14 (vaccinated three times) showed the best induction of IFN-γ-secreting splenocytes.

In contrast, a single priming dose (D14 group) of HBx-preS1-T^{Stx}-18sCD40L did not apparently induce HBx-specific antibody response. However, the second immunization boosted the antibody level moderately (D7-D14 group) and the third dose further boosted the antibody level even higher as shown in the animal group DO-D7-D14 (FIG. 18). The dosing-number-dependent effect in inducing humoral response is consistent with that in inducing cell-mediated immune responses. The fusion protein is effective in inducing IFN-γproduction in a dosing number dependent manner (FIG. 17).

Thus, the fusion protein HBx-preS1-T^{Stx}-18sCD40L could effectively elicit HBx-specific T cell-mediated immune response and HBx-specific humoral immune response after twice immunizations, which could be further boosted by multiple vaccinations.

The foregoing description of the exemplary embodiments has been presented only for the purposes of illustration and description and is not intended to be exhaustive to limit the invention to the precise forms disclosed. All references cited and discussed in this specification are incorporated herein by reference in their entireties and to the same extent as if each reference was individually incorporated by reference.

What is claimed is:

1. A fusion protein comprising:
(a) a CD40-binding domain, which is a CD40 ligand (CD40L) or a functional fragment thereof comprising an amino acid sequence that is at least 90% identical to SEQ ID NO: 19, said CD40L or functional fragment thereof consists of 154-261 amino acids in length;
(b) an antigen, which is a tumor antigen or an antigen of a pathogen, being located at the N-terminal of the fusion protein;
(c) a translocation domain, located between the CD40-binding domain and the antigen, said translocation domain being a Shiga toxin (Stx) translocation peptide; and
(d) a furin and/or cathepsin L cleavage site, located between the CD40-binding domain and the translocation domain.

2. The fusion protein of claim 1, wherein the Stx translocation peptide comprises an amino acid sequence that is at least 90% identical to the amino acid sequence selected from the group consisting of SEQ ID NOs: 12, 13, 14, 15 and 16, with 8-84 amino acid residues in length.

3. The fusion protein of claim 1, wherein the Stx translocation peptide comprises an amino acid sequence of SEQ ID NO: 12 with 8-84 amino acids in length.

4. The fusion protein of claim 1, wherein the furin and/or cathepsin L cleavage site permits removal of the CD40-binding domain away from the fusion protein via furin and/or cathepsin L cleavage.

5. The fusion protein of claim 1, wherein the furin and/or cathepsin L cleavage site comprises an amino acid sequence of SEQ ID NO: 1 or 2.

6. The fusion protein of claim 1, further comprising a peptide linker, said peptide linker comprising the furin and/or cathepsin L cleavage site located between the CD40-binding domain and the translocation domain.

7. The fusion protein of claim 1, wherein the antigen is a tumor antigen, said tumor being selected from the group consisting of breast cancer, colon cancer, rectal cancer, bladder cancer, endometrial cancer, kidney cancer, gastric cancer, glioblastoma, hepatocellular carcinoma, bile duct cancer, small cell lung cancer, non-small cell lung cancer (NSCLC), melanoma, ovarian cancer, cervical cancer, pancreatic cancer, prostate cancer, acute myelogenous leukemia (AML), chronic myelogenous leukemia (CML), non-Hodgkin's lymphoma, and thyroid cancer.

8. The fusion protein of claim 1, wherein the antigen is an antigen of a pathogen, said pathogen being selected from the group consisting of Human Papillomavirus (HPV), Human Immunodeficiency Virus-1 (HIV-1), Influenza Virus, Dengue Virus, Hepatitis A Virus (HAV), Hepatitis B Virus (HBV), Hepatitis C Virus (HCV), Hepatitis D Virus (HDV), Hepatitis E Virus (HEV), Severe acute respiratory syndrome-associated coronavirus (SARS-COV), Severe acute respiratory syndrome coronavirus 2 (SARS-COV-2), Middle East respiratory syndrome coronavirus (MERS-COV), Epstein-Barr virus (EBV), Zika Virus, Rabies Virus, Variola virus, Chikungunya Virus, West Nile virus, Poliovirus, Measles virus, Rubella virus, Hantavirus, Japanese encephalitis virus, Coxsackievirus, Echovirus, Enterovirus, Mumps virus, Varicella-zoster virus (VZV), Cercopithecine herpesvirus-1 (CHV-1), Yellow fever virus (YFV), Rift Valley Fever Virus, Lassa virus, Marburg virus, Ebolavirus, Norovirus, Rotavirus, Adenovirus, Sapovirus, Astrovirus, Rickettsia prowazekii, Rickettsia typhi, Orientia tsutsugamushi, Borrelia burgdorferi, Yersinia pestis, Plasmodium vivax, Plasmodium malariae, Plasmodium falciparum, Plasmodium ovale, Bacillus anthracis, Clostridium Difficile, Clostridium Botulinum, Corynebacterium diphtheriae, Salmonella enterica serovar Typhi, Salmonella enterica serovar Paratyphi A, Shiga toxin-producing E. coli (STEC), Shigella dysenteriae, Shigella flexneri, Shigella boydii, Shigella sonnei, Entamoeba histolytica, Vibrio cholerae, Mycobacterium tuberculosis, Neisseria meningitidis, Bordetella pertusis, Haemophilus influenzae type B (HiB), Clostridium tetani, Listeria monocytogenes and Streptococcus pneumoniae.

9. The fusion protein of claim 2, wherein the Stx translocation peptide comprises an amino acid sequence selected from the group consisting of SEQ ID NOs: 12, 13, 14, 15 and 16.

10. A fusion protein comprising:
(a) a CD40-binding domain, which is a CD40 ligand (CD40L) or a functional fragment thereof comprising an amino acid sequence that is at least 90% identical to SEQ ID NO:
19, said CD40L or functional fragment thereof consists of 154-261 amino acids in length;
(b) an antigen, which is a tumor antigen or an antigen of a pathogen, being located at the N-terminal of the fusion protein;
(c) a translocation domain, located between the CD40-binding domain and the antigen, said translocation domain being a Shiga toxin (Stx) translocation peptide, comprising an amino acid sequence of SEQ ID NO: 12; and
(d) a furin and/or cathepsin L cleavage site, located between the CD40-binding domain and the translocation domain.

11. The fusion protein of claim 10, wherein the Stx translocation peptide comprises an amino acid sequence selected from the group consisting of SEQ ID NOs: 12, 13, 14, 15 and 16.

12. The fusion protein of claim 10, wherein the CD40L or the functional fragment thereof comprises the amino acid sequence of SEQ ID NO: 18 or SEQ ID NO: 19.

13. The fusion protein of claim 1, wherein the CD40L or the functional fragment thereof comprises the amino acid sequence of SEQ ID NO: 18 or SEQ ID NO: 19.

14. The fusion protein of claim 1, wherein the CD40-binding domain or the functional fragment thereof consists of the amino acid sequence of SEQ ID NO: 19.

15. The fusion protein of claim 1, which comprises the amino acid sequence selected from the group consisting of SEQ ID NOs: 53, 54 and 55.

16. The fusion protein of claim 10, which comprises the amino acid sequence selected from the group consisting of SEQ ID NOs: 53, 54 and 55.

17. A method for eliciting an antigen-specific cell-mediated immune response, comprising:
administering a therapeutically effective amount of the fusion protein of claim 1 to a subject in need thereof, and thereby eliciting the antigen-specific cell-mediated immune response in the subject in need thereof.

18. A method for eliciting an antigen-specific cell-mediated immune response, comprising:
administering a therapeutically effective amount of the fusion protein of claim 10 to a subject in need thereof, and thereby eliciting the antigen-specific cell-mediated immune response in the subject in need thereof.

19. A method for treating a tumor or a disease caused by a pathogen in a subject in need thereof, comprising:
administering to the subject in need thereof a therapeutically effective amount of the fusion protein of claim 1, wherein the antigen of the fusion protein is the tumor antigen, and thereby treating the subject in need thereof.

20. A method for treating a tumor or a disease caused by a pathogen in a subject in need thereof, comprising:
administering to the subject in need thereof a therapeutically effective amount of the fusion protein of claim 10, wherein the antigen of the fusion protein is the antigen of the pathogen, and thereby treating the disease caused by the pathogen.


SEQUENCE LISTING (56 sequences)

SEQ ID NO: 1-2 - skipped (2 entries)

SEQ ID NO: 3 - protein, 18 aa, synthetic construct
  REGION 1..18: note = Rigid linker 1
EAAAAKEAAAAKEAAAAK

SEQ ID NO: 4 - protein, 613 aa, Pseudomonas aeruginosa
  REGION 1..613: note = Full length Pseudomonas Exotoxin A (PE)
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 5 - protein, 26 aa, synthetic construct
  REGION 1..26: note = PE translocation peptide (PE280-305, minimal)
GWEQLEQCGYPVQRLVALYLAARLSW

SEQ ID NO: 6 - protein, 34 aa, synthetic construct
  REGION 1..34: note = PE translocation peptide (PE280-313)
GWEQLEQCGYPVQRLVALYLAARLSWNQVDQVIR

SEQ ID NO: 7 - protein, 46 aa, synthetic construct
  REGION 1..46: note = PE translocation peptide (PE268-313)
PLETFTRHRQPRGWEQLEQCGYPVQRLVALYLAARLSWNQVDQVIR

SEQ ID NO: 8 - protein, 61 aa, synthetic construct
  REGION 1..61: note = PE translocation peptide (PE253-313)
GGSLAALTAHQACHLPLETFTRHRQPRGWEQLEQCGYPVQRLVALYLAARLSWNQVDQVIR

SEQ ID NO: 9 - protein, 112 aa, synthetic construct
  REGION 1..112: note = PE translocation peptide (PE253-364)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAN

SEQ ID NO: 10 - protein, 293 aa, Shigella dysenteriae
  REGION 1..293: note = Full length Shiga toxin (Stx) subunit A
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 11 - protein, 293 aa, Escherichia coli
  REGION 1..293: note = Full length Shiga-like toxin I (Slt-I) subunit A
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 12 - protein, 8 aa, synthetic construct
  REGION 1..8: note = Stx translocation peptide (Stx240-247, minimal)
LNCHHHAS

SEQ ID NO: 13 - protein, 12 aa, synthetic construct
  REGION 1..12: note = Stx translocation peptide (Stx240-251)
LNCHHHASRVAR

SEQ ID NO: 14 - protein, 37 aa, synthetic construct
  REGION 1..37: note = Stx translocation peptide (Stx211-247)
PDYHGQDSVRVGRISFGSINAILGSVALILNCHHHAS

SEQ ID NO: 15 - protein, 41 aa, synthetic construct
  REGION 1..41: note = Stx translocation peptide (Stx211-251)
PDYHGQDSVRVGRISFGSINAILGSVALILNCHHHASRVAR

SEQ ID NO: 16 - protein, 84 aa, synthetic construct
  REGION 1..84: note = Stx translocation peptide (Stx168-251)
ALRFRQIQRGFRTTLDDLSGRSYVMTAEDVDLTLNWGRLSSVLPDYHGQDSVRVGRISFGSINAILGSVALILNCHHHASRVAR

SEQ ID NO: 17 - protein, 261 aa, Homo sapiens
  REGION 1..261: note = Full length CD40 ligand (CD40L1-261)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - protein, 215 aa, synthetic construct
  REGION 1..215: note = Truncated CD40 ligand (CD40L47-261)
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 19 - protein, 154 aa, synthetic construct
  REGION 1..154: note = Truncated CD40 ligand (CD40L108-261, also referred to as 18sCD40L)
ENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 246 aa, synthetic construct
  REGION 1..246: note = Anti-CD40 scFv (VH-L-VL)
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 21 - protein, 246 aa, synthetic construct
  REGION 1..246: note = Anti-CD40 scFv (VL-L-VH)
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGGGGGSGGGGSGGGGSEVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 22 - protein, 119 aa, synthetic construct
  REGION 1..119: note = VH of the anti-CD40 scFv
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 23 - protein, 112 aa, synthetic construct
  REGION 1..112: note = VL of the anti-CD40 scFv
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 24 - protein, 10 aa, synthetic construct
  REGION 1..10: note = VH CDR1
GFTFSTYGMH

SEQ ID NO: 25 - protein, 26 aa, synthetic construct
  REGION 1..26: note = VH CDR2
GKGLEWLSYISGGSSYIFYADSVRGR

SEQ ID NO: 26 - protein, 13 aa, synthetic construct
  REGION 1..13: note = VH CDR3
CARILRGGSGMDL

SEQ ID NO: 27 - protein, 15 aa, synthetic construct
  REGION 1..15: note = VL CDR1
CTGSSSNIGAGYNVY

SEQ ID NO: 28 - protein, 7 aa, synthetic construct
  REGION 1..7: note = VL CDR2
GNINRPS

SEQ ID NO: 29 - protein, 12 aa, synthetic construct
  REGION 1..12: note = VL CDR3
CAAWDKSISGLV

SEQ ID NO: 30 - protein, 4 aa, synthetic construct
  REGION 1..4: note = ER retention sequence
KDEL

SEQ ID NO: 31 - protein, 12 aa, synthetic construct
  REGION 1..12: note = ER retention sequence
KKDLRDELKDEL

SEQ ID NO: 32 - protein, 13 aa, synthetic construct
  REGION 1..13: note = ER retention sequence
KKDELRDELKDEL

SEQ ID NO: 33 - protein, 13 aa, synthetic construct
  REGION 1..13: note = ER retention sequence
KKDELRVELKDEL

SEQ ID NO: 34 - protein, 12 aa, synthetic construct
  REGION 1..12: note = ER retention sequence
KDELKDELKDEL

SEQ ID NO: 35 - protein, 28 aa, synthetic construct
  REGION 1..28: note = CD28 consensus sequence
  VAR_SEQ 8: note = X is I or L
  VAR_SEQ 10: note = X is V, F or A
  VAR_SEQ 11: note = X is M or L
  VAR_SEQ 17: note = X is L or I
TDIYFCKXEXXYPPPYXDNEKSNGTIIH

SEQ ID NO: 36 - protein, 28 aa, synthetic construct
  REGION 1..28: note = CD28-activating peptide (minimal)
TDIYFCKIEVMYPPPYLDNEKSNGTIIH

SEQ ID NO: 37 - protein, 53 aa, synthetic construct
  REGION 1..53: note = CD28-activating peptide
NCDGKLGNESVTFYLQNLYVNQTDIYFCKIEVMYPPPYLDNEKSNGTIIHVKG

SEQ ID NO: 38 - protein, 98 aa, Human papillomavirus type 16
  REGION 1..98: note = Antigen HPV16 E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 39 - protein, 104 aa, Human papillomavirus type 18
  REGION 1..104: note = Antigen HPV18 E7 protein
HGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 40 - protein, 154 aa, Hepatitis B virus
  REGION 1..154: note = Antigen HBV X protein (HBx; full length)
MAARMCCQLDPARDVLCLRPVGAESRGRPLPGPLGALPPSSASAVPADHGSHLSLRGLPVCSFSSAGPCALRFTSARRMETTVNAPWSLPTVLHKRTIGLSGRSMTWIEEYIKDCVFKDWEELGEEIRLKVFVLGGCRHKLVCSPAPCNFFTSA

SEQ ID NO: 41 - protein, 108 aa, Hepatitis B virus
  REGION 1..108: note = Antigen HBV preS1 protein
MGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPI

SEQ ID NO: 42 - protein, 190 aa, Hepatitis C virus
  REGION 1..190: note = Antigen HCV core protein (full length)
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPAS

SEQ ID NO: 43 - protein, 1273 aa, Severe acute respiratory syndrome coronavirus 2 (SARS-CoV-2)
  REGION 1..1273: note = Antigen SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 187 aa, Homo sapiens
  REGION 1..187: note = Antigen SSX2
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDD

SEQ ID NO: 45 - protein, 314 aa, Homo sapiens
  REGION 1..314: note = Antigen MAGE-A3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 46 - protein, 180 aa, Homo sapiens
  REGION 1..180: note = Antigen NY-ESO-1
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 47 - protein, 296 aa, Homo sapiens
  REGION 1..296: note = Antigen iLRP
FSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 48 - protein, 279 aa, Homo sapiens
  REGION 1..279: note = Antigen WT12-281
GSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPI

SEQ ID NO: 49 - protein, 406 aa, Homo sapiens
  REGION 1..406: note = Antigen RNF43
SGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPAHPLICGPPGLDKRLLPETPGPCYSNSQPVWLCLTPRQPLEPHPPGEGPSEWSSDTAEGRPCPYPHCQVLSAQPGSEEELEELCEQAVSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 50 - protein, 284 aa, Homo sapiens
  REGION 1..284: note = Antigen CEA-NE3
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDTPIISPPDSSYLSGANLNLSCHSASNPSPQYSWFVNGTFQQHTQVLLIAKIQPNNNGTYACFVSNLATGRNNSIVKSITV

SEQ ID NO: 51 - protein, 528 aa, synthetic construct
  REGION 1..528: note = Fusion protein CD40L47-261-TPE-E7
MHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAKDEL

SEQ ID NO: 52 - protein, 467 aa, synthetic construct
  REGION 1..467: note = Fusion protein 18sCD40L-TPE-E7
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAKDEL

SEQ ID NO: 53 - protein, 535 aa, synthetic construct
  REGION 1..535: note = Fusion protein E7-TStx-CD40L47-261
MKLTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAPDYHGQDSVRVGRISFGSINAILGSVALILNCHHHASRVAREFGSELEAAAAKEAAAAKEAAAAKHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 54 - protein, 474 aa, synthetic construct
  REGION 1..474: note = Fusion protein E7-TStx-18sCD40L
MKLTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 55 - protein, 541 aa, synthetic construct
  REGION 1..541: note = Fusion protein HBx-preS1-TStx-18sCD40L
MKLTMGSSHHHHHHSSGLVPRGSHMAARMCCQLDPARDVLCLRPVGAESRGRPLPGPLGALPPSSASAVPADHGSHLSLRGLPVCSFSSAGPCALRFTSARRMETTVNAPWSLPTVLHKRTIGLSGRSMTWIEEYIKDCVFKDWEELGEEIRLKVFVLGGCRHKLVCSPAPCNFFTSARGRAEAAAAKEAAAAKEAAAAKEAAAAKAEFMGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPIELTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 56 - protein, 6 aa, synthetic construct
  REGION 1..6: note = Rigid linker 1 with n=1
EAAAAK